(19) United States
(12) Patent Application Publication
(10) Pub. No.: US 2006/0165717 A1
(43) Pub. Date: Jul. 27, 2006

(54) DCchol in newborns

(75) Inventors: Francois Dalencon, Lyon (FR); Claire-Anne Siegrist, Geneva (CH)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Sanofi Pasteur

(21) Appl. No.: 11/338,908

(22) Filed: Jan. 24, 2006

Related U.S. Application Data
(60) Provisional application No. 60/671,195, filed on Apr. 14, 2005.

(30) Foreign Application Priority Data
Jan. 25, 2005 (FR) 05 00743

(51) Int. Cl. A61K 39/295; A61K 39/02

(52) U.S. Cl. 424/190.1; 424/202.1

(57) ABSTRACT

The present invention involves the use of DCchol for preparing a vaccine composition intended to be administered to newborns.

### BACKGROUND OF THE INVENTION

The present invention relates to the use of a vaccine adjuvant for preparing a vaccine intended for administration to newborn mammals, and also to a method of immunizing newborns. In particular, the invention relates to the use of DCchol for preparing a vaccine for newborns.

The development of vaccines is always difficult, but it is particularly complicated when it involves vaccines intended for newborns; specifically, it has been noted that, when immunization is carried out during the 1st month of life, a smaller amount of antibodies is obtained than when the immunization is carried out later; in addition, the antibodies obtained generally have a shorter lifetime than when the immunization takes place later.

Furthermore, it would seem that the T-cell responses obtained at an early stage in life, although they are responses that mature rapidly, are responses that are both qualitatively and quantitatively different from those that are induced at a subsequent stage in life.

However, newborns would need to be armed very early on, in order to be able to combat a certain number of pathogens to which they may be exposed while very young, in particular the human immunodeficiency virus, the respiratory syncytial virus, the agent for malaria, Helicobacter pylori, Haemophilus influenza type B, the poliovirus, the hepatitis B virus, and the agents for whooping cough, for tetanus, for diphtheria, for meningitis and for pneumonia.

Consequently, there exists a great need to be able to provide vaccine compositions for effectively immunizing newborns.

### BRIEF DESCRIPTION OF THE INVENTION

In order to satisfy this need, a subject of the present invention is the use of DCchol for preparing a vaccine composition intended to be administered to newborns.

### BRIEF DESCRIPTION OF THE SEVERAL VIEWS OF THE DRAWING

FIG. 1 shows the total amount of IgG antibody expressed in log₁₀ of the arbitrary ELISA titers, as described below.

### DETAILED DESCRIPTION OF THE INVENTION

According to a particular embodiment, the vaccine composition comprises at least one antigen against infections caused by Helicobacter pylori.

According to another particular embodiment, the vaccine composition comprises at least the TAT antigen of the AIDS virus.

A subject of the invention is also a method of immunizing newborns, according to which at least one vaccine antigen is administered to newborns, in which the improvement consists in also administering at least one adjuvant comprising DCchol.

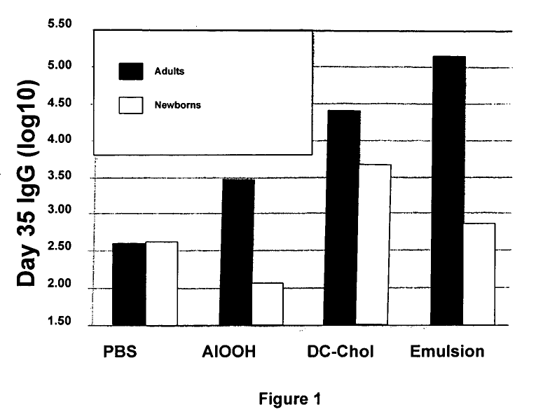

Many advantages of the present invention will emerge on reading the detailed description that follows, with reference to FIG. 1 which illustrates the results obtained in Example 1.

For the purposes of the present invention, the term “newborns” is intended to mean infants that are at most one month old at the time that they receive the first injection.

Given the fact that it is desirable to reduce as much as possible the clinical trials carried out in humans, and in particular in infants and newborns, an animal model has been developed. In fact, although it is difficult to strictly compare immunomaturation from one species to another, many experiments have, however, made it possible to validate the hypothesis that, whatever the species, the immunomaturation always follows the same steps, the difference between the various species lying in the kinetics of passage from one step to the other. Thus, the vaccine formulations according to the present invention, although essentially intended for administration to human newborns, were developed on the basis of tests carried out on young mice, the steps of immunomaturation of the young mouse to the adult mouse having been demonstrated as being essentially the same as the steps of immuno-maturation of the human newborn to the adult human. In this model, human newborns one month old are represented by young mice one week old.

According to the invention, the expression “enhancement of the immune response” is intended to mean the obtaining, in the newborn, of an immune response that is similar to that obtained in adults; it may in particular be an antibody response or a T-cell response, which would be quantitatively increased compared with that generally obtained with the vaccines of the prior art, or else a qualitative modification of the response obtained; the qualitative characteristics of the immune responses possibly being expressed in terms of the nature of the antibodies produced, and also in terms of the nature and the amounts of the cytokines induced, which makes it possible in particular to determine the Th1 or Th2 orientation of the response.

In mice, when the humoral response is studied, i.e. when the immunoglobulins produced are studied, it is considered that IgG2 as reflect a Th1-oriented response, and that IgG1s reflect a Th2-oriented response. When the cytokines produced are examined, it is considered that interferon-γ indicates a Th1 orientation, whereas interleukins 4 and 5 indicate a Th2 orientation.

For the purpose of the present invention, the term “DCchol” is intended to mean 3β-[N-(N′,N′-dimethyl-aminoethane)carbamoyl]cholesterol, which can be obtained from cholesteryl chloroformate and N,N-dimethylethylenediamine, according to the method described in U.S. Pat. No. 5,283,185 or else according to the method described in Example 8 of U.S. Pat. No. 5,753,262. Preferably, the saline form of DCchol, in particular the chloride, is used.

The term “vaccine composition” is intended to mean a composition comprising at least one vaccine antigen of interest for the immunization of newborns. It may be a monovalent vaccine composition, i.e. comprising a single type of antigen, or a vaccine combination comprising several antigens for simultaneously immunizing against one or more diseases.

The antigens which may be adjuvanted by means of the present invention may be diverse in nature; they may be proteins, peptides, oligosaccharides or polysaccharides (conjugated or unconjugated), viruses or portions of viruses, etc., more generally any advantageous antigen to be introduced into a pediatric vaccine composition.

The present invention does not concern the use of DCchol for adjuvanting vaccine compositions which, instead of comprising the antigens themselves, comprise the antigens in the form of their “precursor”, namely DNA.

The present invention is of particular advantage in the case of certain diseases capable of affecting a newborn from the first moments of its life; this involves, for example, AIDS, malaria, etc.

According to the invention, the vaccine composition can be administered by any of the routes normally used for administering vaccines, and in particular subcutaneously, intramuscularly, transcutaneously and intradermally.

The administration scheme is advantageously a scheme that provides for at least 2 successive administrations: a 1st administration followed by a booster some time later, the 1st administration being carried out in the 1st month of life.

Advantageously, the vaccine composition prepared according to the invention exhibits increased immuno-genicity without, however, experiencing increased reactogenicity.

The reactogenicity can be assessed in various ways; in mice, the following monitoring is carried out:
local reactogenicity: at the site of injection, verification of the presence or absence of an inflammatory granuloma with the presence or absence of a scar that prevents hair growth,
systemic reactogenicity: daily weighing to verify whether or not weight gain is normal.

Conventionally, the vaccine compositions according to the invention can also comprise any of the elements sometimes present in the compositions according to the prior art, such as preserving agents, stabilizers, or the like.

The following examples illustrate some embodiments of the present invention.

1. Vaccine Composition Comprising the TaT Protein

1.1 Preparation of the Vaccine Compositions

Vaccine compositions comprising, as antigen, a detoxified TAT III B protein are prepared. The TAT protein was detoxified by means of an alkylation reaction in an alkaline medium using iodoacetamide under the following conditions: number of micromoles of iodoacetamide=200×number of micromoles of TAT+number of micromoles of DTT. This detoxified protein and the method for preparing it are described in detail in application WO 99/33346, where it is identified under the term “carboxymethylated TAT”.

This recombinant TAT antigen is present in solution at 2 g/l.

The vaccine compositions are prepared either with an adjuvant according to the prior art, i.e. aluminum oxyhydroxide or a squalene-based emulsion, or according to the invention with DCchol; in addition, in order to be able to demonstrate the adjuvant capacity of the various products tested, a composition comprising only the antigen in saline solution is also prepared.

The composition comprising aluminum oxyhydride is prepared from a suspension at 8 g/l, provided by the company Superfos Biosector.

The emulsion is prepared by mixing, in an Ultraturrax for a few seconds, 0.18 g of Tween™ 80 (provided by Merck), 0.16 g of sorbitan trioleate (provided by the company Seppic under the name Montane VG™85) and 1.29 g of squalene (provided by the company Fluka) in a sufficient amount of water for 30 ml. A pre-emulsion is therefore obtained, which is homogenized in a microfluidizer M110 (Microfluidics, Newton Mass.) by means of 20 passages at a motor pressure of 60 psi to give a monodisperse emulsion with droplets having a mean size of 120 nm.

The liposomal composition of DCchol is prepared by dispersing 1.3 g of DCchol powder (provided by the company Avanti® Polar Lipids Inc) in 65 ml of distilled water; the suspension is stirred for 15 min at ambient temperature (22° C.) and for 1 hour at 65° C. After cooling to around 39-40° C., the suspension is filtered through a Durapore™ hydrophilic filter (Millipore) with a 0.45 μm porosity, and under pressure through an Anotop™ filter (Whatmann) with a 0.2 μm porosity. A suspension of liposomes is then obtained, the mean size of which is 176 nm, and the concentration of which is 20 μg/ml.

Each of the compositions prepared is diluted in a saline solution which contains a final concentration of 50 mmol/l of tris(hydroxymethyl)aminomethane and 100 mmol/l of sodium chloride, the pH being adjusted to 7.4.

Immunizing doses having the following composition are the obtained:

| Nature of the | Amount of | Amount of | Size of |
|---|---|---|---|
| adjuvant | adjuvant | antigen | the dose |
| None (saline | | 1 μg | 50 μg |
| solution only) | | | |
| AlOOH | 200 μg | 1 μg | 50 μg |
| Emulsion | Squalene: 2.15 mg | 1 μg | 50 μg |
| | Tween ™ 80: 0.3 mg | | |
| | Sorbitan trioleate: 0.26 mg | | |
| DCchol | 100 μg | 1 μg | 50 μg |

1.2 Immunization of the Mice

Adult Balb/c mice and 1-week-old “newborn” Balb/c mice are provided and are divided up into groups of 6 to 8 (groups of young mice) or of 5 to 6 (groups of adult mice). Experiments are carried out with 2 subcutaneous injections of the same vaccine formulation at D0 and at D21. Blood samples are taken after the booster injection. The TAT-specific immunoglobulins are assayed by the ELISA technique. A total of 10 to 40 mice per formulation was tested in several independent experiments: for the DC-Chol: 27 adults, 38 newborns; for the aluminum oxyhydroxide: 18 adults, 22 newborns; for the emulsion: 10 adults and 16 newborns; and in saline solution: 10 adults and 15 newborns.

The results obtained are given in FIG. 1, which indicates, for each of the formulations tested, the total amount of IgG antibody expressed in log₁₀ of the arbitrary ELISA titers.

These results show that the aluminum oxyhydroxide and the emulsion, which are good adjuvants in the adult mice, do not give satisfactory results in the newborns; unlike them, the DCchol is capable of increasing the immune response induced by a vaccine composition when it is administered to adults, but also when it is administered to newborns.

2. Vaccine Composition Comprising Helicobacter pylori Urease

2.1. Preparation of the Vaccine Compositions

Vaccine compositions having, as antigen, recombinant Helicobacter pylori urease are prepared. This urease has already been described as a potential candidate for the production of a vaccine against stomach ulcers induced by the bacterium Helicobacter pylori. A method for preparing it is described in the following publication: Journal of Infectious Diseases, 1995. Jul:172(1):161-72. “Oral immunization with recombinant Helicobacter pylori urease induces secretory IgA antibodies and protects mice from challenge with Helicobacter felis.” Lee C K, Weltzin R, Thomas W D Jr, Kleanthous H, Ermak T H, Soman G, Hill J E, Ackerman S K, Monath T P.

The immunizing compositions are prepared in the same way as described in the previous example, except that, at the time of the final mixing, the suspension comprising the Tat antigen is replaced with a suspension comprising urease. Immunizing doses of 50 μl comprising, each time, 10 μg of urease and either 200 μg of DCchol or 200 μg of AlOOH are obtained.

2.2 Immunization of the Mice

Adult Balb/c mice and 1-week-old Balb/c mice are provided and are divided up into groups of 5 to 8 (groups of adult mice or groups of young mice).

Experiments are carried out with 2 subcutaneous injections of the same vaccine formulation at D0 and at D21 (experiments 1 and 2), or with 3 injections at D0, D21 and D36 (experiments 3 and 4).

Blood samples are taken after the 1st injection, after the 1st booster and, optionally, after the 2nd booster (in the case of experiments 3 and 4).

The urease-specific immunoglobulins are assayed by the ELISA technique.

The results relating to the titers measured, expressed as log₁₀ of the ELISA titers, relating to the amounts of IgG1 and of IgG2a produced in the newborn mice are indicated in the table below:

| | | IgG1 | | | IgG2a | | |
|---|---|---|---|---|---|---|---|
| | | | After | After | After | After | After |
| | | After | 1st | 2nd | 1st | 1st | 2nd |
| | | 1st inj. | booster | booster | inj. | booster | booster |
| Exp. 1 | AlOOH | 2.77 | 3.50 | | 0.87 | 1.08 | |
| | DCchol | 3.74 | 4.89 | | 2.18 | 2.41 | |
| Exp. 2 | AlOOH | 2.40 | 3.26 | | 0.96 | 2.02 | |
| | DCchol | 3.07 | 4.50 | | 1.30 | 2.69 | |
| Exp. 3 | AlOOH | 2.43 | 3.55 | 4.32 | 2.64 | 2.93 | 3.19 |
| | DCchol | 3.16 | 4.36 | 4.73 | 2.70 | 2.78 | 3.55 |
| Exp. 4 | AlOOH | 2.45 | | 3.50 | 1.00 | | 2.33 |
| | DCchol | 3.22 | | 4.64 | 1.79 | | 3.32 |

The assays of the amounts of antibodies present in the young mice before the 1st injection cannot be carried out without sacrificing the animals.

Thus, it is not possible to have, for the same young mouse, its antibody titer before immunization and after immunization; however, a certain number of assays were nevertheless carried out and, although they vary according to the maternal antibodies, it could be observed that they were, on average, less than those obtained after the 1st injection, which clearly makes it possible to validate the effect of the immunization.

The results obtained show that DCchol makes it possible to induce a greater amount of antibodies in the newborns than aluminum oxyhydroxide.

In each of the experiments carried out, although this is not described in detail here, the same number of adult mice was also immunized; the results obtained show that the antibody titers in the adult mice are slightly higher than in the young mice; on the other hand, it is seen, in the adult population, that, although DCchol is as good an adjuvant as aluminum oxyhydroxide, it is not better than it in terms of antibody production.

Furthermore, the criteria for assessing reactogenicity of the vaccine compositions are satisfactory whatever the adjuvant used.

1. The use of DCchol for preparing vaccine compositions intended to be administered to newborns.

2. The use as claimed in claim 1, wherein the vaccine composition comprises at least one antigen against infections caused by Helicobacter pylori.

3. The use as claimed in claim 1, wherein the vaccine composition comprises at least the TAT antigen of the AIDS virus.

4. A method of immunizing newborns, according to which at least one vaccine antigen is administered to newborns, in which the improvement consists in also administering at least one adjuvant comprising DCchol.
